(19) United States
(12) Patent Application Publication
(10) Pub. No.: US 2018/0295767 A1
(43) Pub. Date: Oct. 18, 2018

(54) Unterface Attachment System and Method

(71) Applicant: LANDON JAMES FRIEMEL, HEREFORD, TX (US)

(72) Inventor: LANDON JAMES FRIEMEL, HEREFORD, TX (US)

(21) Appl. No.: 15/950,324

(22) Filed: Apr. 11, 2018

Related U.S. Application Data
(60) Provisional application No. 62/485,522, filed on Apr. 14, 2017.

(51) Int. Cl. A01B 63/02; A01D 84/00; A01B 59/06; A01B 76/00

(52) U.S. Cl. CPC A01B 63/02; A01D 84/00; G01N 33/246; A01B 76/00; A01B 59/06

(57) ABSTRACT

The Unterface system disclosed includes a machine-unique Down-Facing component adapted to connect Ag-machine structures, hydraulics, DC power and CAN network matingly with an attachment-unique Up-Facing component fixed to the attachment whereby the attachment is efficiently, fully and safely operable as an ordered combination with the ag-machine and its various control networks.

### CROSS REFERENCE TO RELATED APPLICATIONS

This application claims the benefit of U.S. Provisional Application No. 62/485522, filed 14 Apr 2017, the contents of which are incorporated herein by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT.

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB).

Not applicable.

### STATEMENT REGARDING PRIOR DISCLOSURES BY THE INVENTOR OR A JOINT INVENTOR.

Not applicable.

### BACKGROUND OF THE INVENTION

### Field of the Invention.

Modern precision agriculture uses high-tech computer systems to maximize crop yields and minimize inputs of labor, equipment and supplies, especially fertilizer, pesticides and irrigation. In 2015 the the global agriculture and farm machinery market was about 144 000 000 000 USD and is forecast to grow at about 9% annually over the coming decade. The world market is served by a very small group of firms; in the US, there is a single dominant firm for precision ag machines and systems.

Generally, swathing or merging attachments for self-propelled ag machines are custom designed by each of the major equipment firms and are not easily interchangeable even over their own legacy models. In case of a breakdown during harvest season, this situation can create serious management problems for small producers operating a few costly machines of differing marques and vintages.

For premium alfalfa harvests, which must be accomplished within a few days during the peribloom stage, a failure of critical components of a header or swather can mean full or substantial loss of crop value, e.g., low protein level, high moisture content, loss of leaves during cutting and baling. Even with advanced over-night logistics for costly new parts and the possible option of 24-7 emergency field-repair service for current machines, every forage producer would welcome the availability of a universal swather mount adapted to work with all brands, models types and ages of machines. While such devices would facilitate emergency rental of attachments from any dealer and/or sharing of equipment between local producers, they would also increase competition with the oligarchic ag-machine industry. Attaching a new-design belly implement or adapting foreign merger for belly mounting on a third-party precision-ag machine is: (a) far from an obvious undertaking for a typical PHOSITA and (b) generally a very expensive shop project including multiple, sophisticated engineering-design challenges, especially network and safety features.

Typically belly-mounted attachments for low-powered and miniature hobby-farm tractors are: rotary mowers, transverse rotary brushes and transverse blade-like implements for landscape leveling or producing a textured, footing surface for training or racing of horses. On a garden tractor, such attachments are typically suspended by fixtures mounted on the extreme front of the frame. The opposite (rear) end of such attachment-support members is usually coupled to the lift arms of the rear three-point hitch (TPH) whereby the operator can achieve lift, or height control of the particular landscaping component. Since a typical precision-ag machine has no TPH and is fitted to mount wide front headers designed for large-scale planting, spraying and harvesting of crops, such configurations are manifestly inapplicable.

### Description of Related Art including information disclosed under 37 CFR 1.97 and 1.98.

Careful searching on the USPTO databases for universal windrower-belly-mounting devices and systems designed for modern self-propelled ag machines revealed neither patent applications nor actual grants which disclose anything remotely similar to the present invention. A single Design grant that was found under word searching for Design and windrower or swather was D243948, granted to Hesston Corp. in 1977. As revealed in FIGS. 2 and 3 of the document, this Design discloses the general configuration_shape and ornamental appearance of a single-axle, 2-wheeled towing tractor_unit pulling a right-side-offset, 2-wheeled implement carrier (which is apparently capable of mounting a header intended for harvest of forage crops). The exemplary figure shows two forward-facing, horizontal bars/tubes (bar axis parallel to travel direction) on the trailed carrier component which apparently are intended to receive or connect to and support some type of a mating header frame. It is not clear if this Design covers a toy or an actual Tournapull-type towed-harvesting-machine trailer having its rotor ground-driven by the wheels. Because many Designs titled as tractor disclose toys and decorative items, this seems a reasonable possibility.

While traditional family-farmers frequently utilize services of a local truck mechanic or blacksmith to repair broken tillage implements and stalled farm tractors, a modern precision-ag machine valued at over 150 000 USD is far too complex to be serviced by any but specially-trained technicians working in a shop provided with a plethora of special tools and digital testing equipment. Further, the liability risks of even simple MIG-welding on a broken steel feature on such complex systems typically is beyond the insurance coverage of all but large nation-wide dealerships. Considering these and a profusion of technical factors, it is irrational to speculate that a typical farmer-owner and local mechanics could design and fabricate hillbilly fixtures to accomplish coupling of attachments designed and sold for one marque onto a machine of a differing brand, function, model, vintage, or type. Moreover, even if such a MacGyver approach was apparently successful after a few days of early testing and revisions, there would be no repair parts for future in-service emergencies including wearout/breakdowns of the resulting one-off fixture.

### BRIEF SUMMARY OF THE INVENTION

The Unterface attachment-mounting system is a novel apparatus for coupling attachments to an ag machine and can become the basis for industry-standards for belly mounting of machine-and-crop-interoperable harvesting attachments.

### BRIEF DESCRIPTION OF THE DRAWINGS

FIG. 1A. Typical Machine, Left Side View

FIG. 1A. shows the left side (machine forward direction to the left) for the JD W235;

FIG. 1B Typical Four-Bar Actuator for Cross-Belt Swather Attachment

FIG. 1B shows an oblique exterior view of a typical actuator for under-belly attachments.

Dimensional data for the JD W235 Windrower (Weight =7303 kg) include: Overall length without platform 5,900 mm; Overall height 3,760 mm; Under frame clearance 1,120 mm; Wheelbase 3,400 mm; Tread width 3,710 mm.

FIG. 2A. Mounted Merger in Up State, Right Side View

FIG. 2A. is a schematic view from the right side of the merger mounted on a typical machine (machine travel direction to the right) showing the pitch angle of the cross belt in the Up-state;

FIG. 2B. Top View

FIG. 2B. is a broken-out schematic top view of the typical machine showing the Unterface of the present invention, a mounted merger and the yaw angle of the cross belt in the Up-state.

FIG. 3A Right Side View

FIG. 3A is a schematic view from the right side of the merger mounted on a typical machine (machine travel direction to the right) showing the pitch angle of the cross belt in the Down state;

FIG. 3B. Rear View

FIG. 3B. is a broken-out schematic rear view of the typical machine showing the Unterface of the present invention, a mounted merger and the roll angle of the cross belt in the Down state;

FIG. 3C. Top View

FIG. 3C. is a is a broken-out schematic top view of the typical machine (travel direction to the right) showing the Unterface of the present invention, a mounted merger, the yaw angle of the cross belt in the Down state and the yaw angle of the typical attachment actuator re the machine centerline.

FIG. 4A. Actuator Primitive

FIG. 4A. shows a schematic oblique view of the top, end and side planes of the actuator primitive swept out by full-travel of the four-bar linkage (Up- to Down-states);

FIG. 4B. Oblique View Looking Forward

FIG. 4B. shows a schematic 3D perspective view the combined swept volume of the actuator and merger in their movements;

FIG. 4C. (dashed lines) shows a schematic 3D perspective view of the un-occupied volume available for the Unterface primitive (both elements) below the machine frame, i.e., the space above the actuator mounting frame;

FIG. 4D. Right Side View

FIG. 4D. is a schematic 3D right-side view of the swept volume of the actuator, merger and Unterface primitive shown in FIG. 4B. above; this figure also indicates the ground clearance of the actuator and cross belt in Down state.

FIG. 5A. Top View

FIG. 5A. is a broken-out schematic top view showing the actuator and cross belt in the down state; this view also shows the shield erected to re-direct crop flow and the Unterface of the present invention;

FIG. 5B. Oblique Schematic View Showing Frame Rails and UP

FIG. 5B. shows schematic oblique views of a typical machine channel frame and the Unterface primitive; by the use of custom-shaped U-bolts and spacer fittings, the Unterface system can be adapted to machine frames which utilize steel structural tubing in round, square and rectangular shapes as an alternative to classic channel sections.

FIG. 6A. An Alternative Unterface Up-Facing Element Configuration.

FIG. 6A. is a schematic 3D oblique diagram showing one Unterface-System alternative adapted to mount a foreign merger which was provided originally with flange-suspended actuator for the cross belt. The alternative Down-facing and Up-facing elements are shown for this configuration; this specific Up-facing element includes two angle-type transversal components which attach to the actuator. Depending upon the specific attachment, Up-facing transversal component(s) may be one or more of: C- or U-channel, equal or unequal-L angle sections, hollow sq-box or rectangle sections, weldments formed of one or more hot-formed profiles or a completely bespoke section. The nomenclature transversal means generally transverse to the machine axis when the attachment is mounted for use. At coupling to the machine, their tips are affixed to the peripheral ribbon of the Down-facing plate with a set of appropriate high-strength fasteners. The Up-Facing element may also include an optional unique array of ground-end, stabilizing spacers to be threaded on the set of matching fasteners which connects the attachment to the Down-Facing plate element. This array distributes accidental overloads applied to the cross belt and actuator across multiple high-strength fasteners and avoids shear-tear-out at bolt holes close to the edge. FIG. 6B. Another Alternative Unterface Up-Facing Element Configuration.

FIG. 6B. is a schematic 3D oblique diagram shows another Unterface-System alternative adapted to mount a foreign merger provided with a frame-mounted actuator to a machine with specific-tall ground clearance of the frame, i.e., the UP thickness for the particular machine-attachment combination is more than about 30 mm and less than about 100 mm. As shown, the spacer lengths and depth of the Up-facing transversal component are adapted to the particular situation (see L181 and L184 of the Example).

FIG. 7A. Schematic Top View

FIG. 7A. is a schematic of Unterface elements showing profiles of: Down-Facing main plate, displaced Up-Facing element, the two displaced, un-assembled stiffener components, the dashed outline of the actuator mounting frame along with typical locations of inter-element fasteners which couple the Down- and Up-facing elements (see FIG. 8H and 117). The center-lines indicate mating holes for the fasteners, 17 or 117, which couple the Up-facing transversal component and Down-facing plate.

FIG. 7B. Schematic Oblique View

FIG. 7B. is an Oblique View of complete Down-Facing Element (showing weldment with front and rear stiffeners attached and including frame-attachment U-bolts). In this view, both stiffeners are shown after welding into their final position.

FIG. 8A. Unterface System Example for Commercial Machine and Foreign Windrower

FIG. 8A. is an oblique-view rendering of the merger attachment along with the un-connected MacJohn Down-facing element correctly positioned and above the actuator mounting frame.

FIG. 8B. Schematic Top View showing bolt holes and other detail

FIG. 8C Schematic Top View showing front stiffener and other detail

FIG. 8D Schematic Top View showing aft stiffener and other detail

FIG. 8E Schematic Side View showing length of Down-Facing element

FIG. 8F Schematic Side View showing details of aft stiffener

The schematic front view FIG. 8F is an enlarged, broken-out detail of the aft stiffener showing its height and thickness.

FIG. 8G Schematic Side View with broken section showing details of front and aft stiffeners

The schematic right view FIG. 8G includes a broken-away portion of the aft stiffener to expose the height of the forward stiffener. TABLE 2 indicates the value range of each dimension anticipating undocumented variations in parameters of the attachment along with “drift” of machine frame characteristics due to changes over time in manufacturing procedures and suppliers.

FIG. 8H. Schematic Oblique Exploded View of elements

FIG. 8H. is a schematic exploded view of MacJohn elements correctly positioned above the actuator mounting frame of the attachment. Also included are a perspective view of the MacJohn Up-facing element and the transversal component along with its schematic cross-section.

FIG. 8I. Cross-Section View of Up-Facing Element

FIG. 8J. Oblique View of Up-Facing Element

### DETAILED DESCRIPTION OF THE INVENTION.

The “Unterface system” of the present invention provides the possibility to utilize a wide range of attachments with differing mounting-base configurations by a self-propelled, 4-wheeled ag machine. The present invention provides a universal mounting-coupling system, i.e., Down- and Up-Facing elements, engagement features and coupling connections, to link an attachment actuator (or other mounting base) to the machine frame at a particular 3D location which provides power and required operational spaces for the actuator and other crop-management features such as the transverse belt on a merger. TABLES 1 and 2 provide full, clear, concise, and exact technical data on the invention disclosed.

As used herein, the coined word (noun) Unterface means the combination of a machine-unique Down-Facing component adapted to connect machine structures, hydraulics, DC power and CAN network matingly with an attachment-unique Up-Facing component fixed to the attachment whereby the attachment is efficiently, fully and safely interconnected and operable as an ordered combination with the machine.

The systems-mating features of the Unterface include: mechanical-mounting, driving/manipulation power links, electrical links, electronic/network control links, hydraulic links and alarms-status display coordination between all the connected components. The several systems-mating Unterface engagement features are configured congruently for all Down-and Up-Facing parts so that a single Unterface Down-Facing component fixed on a particular machine can adapt and connect effectively with functionally-mated Up-Facing components fixed on several different specialized attachments.

Typical modern self-propelled ag machines are fitted with an array of hydraulic motors able to steer and/or drive each wheel independently and do not have rigid axles transverse to the axis which may obstruct certain attachments. As used herein, the nomenclature frame_rail means the portions of machine structures which extend under the cab and between the four wheels, usually in the form of interconnected, robust steel channel sections which form a ladder frame. Generally, this belly area under such machines is completely available for installation and operation of an attachment or merger using the Unterface system of the present invention. Attachment of a Down-Facing Unterface element to a frame_rail of the machine is done with known, high-strength fasteners and alternative -devices, -technics considering the mechanical-strength standards required for ag or industrial machines. The plate-like elements of the Unterface Down-Facing element are formed and/or fabricated typically as weldments from known materials (usually steels) of size, stiffness and strength adequate to support the attachment and its in-use loads. Unterface fastening technics, including fastener material, size, count, tightening torque and spacing patterns are adapted to comply with standard engineering guidelines.

The vector-angle nomenclature convention used herein is the typical default for engineering drawings, i.e., horizontal=0 degrees and rotation of the vector CCW to vertical is is defined as a +90 degrees. Particular vector parameters are given from the coordinate origin and denoted in decimal degrees, polar form, 0-360. Angles defined on drawings are specified according to std. drafting conventions.

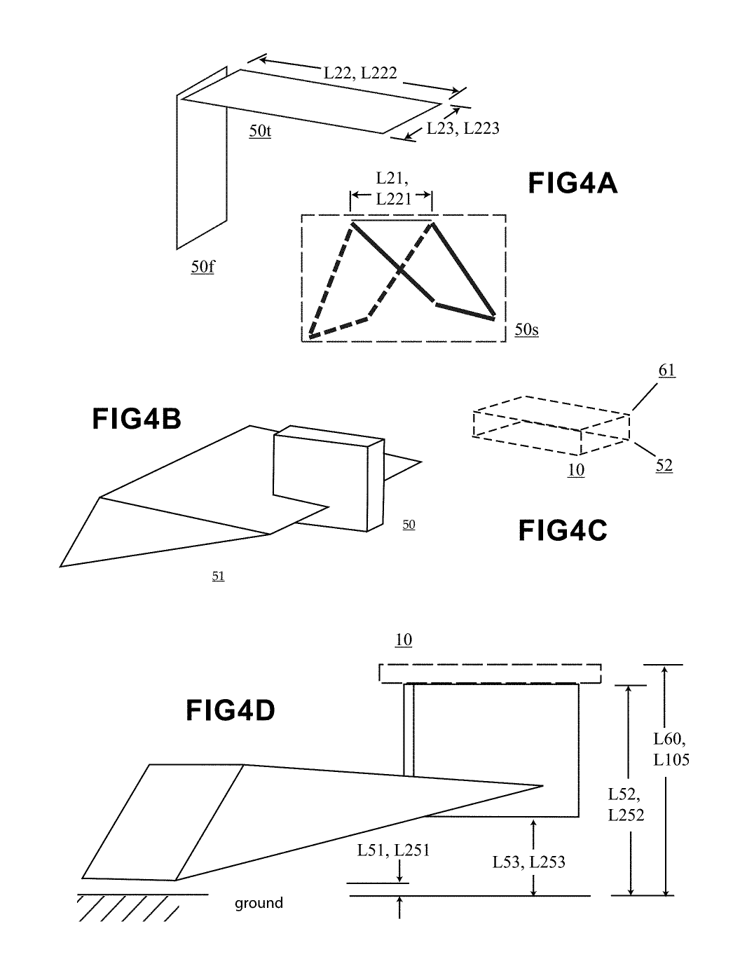

As used herein, attachment 3D loft-volume-space (LVS) is the 3D spatial volume which must be available between the Up and Down states generated by the attachment when the actuator as properly mounted under the machine. Generally an attachment or merger is used for crop management only in the full-Down state. For a Down merger, the ground clearance of both the actuator bottom portion and the distal tip of the cross belt are typically fixed by the original design; this means that the mounting arrangements for a different machine must consider these as requirements to be accommodated by the Unterface system. The LVS is a 3D-loft since horizontal cross-section shapes across and along through the fully-extended attachment (usually the Down state) vary widely between the top and bottom horizontal slices for the Up and Down states respectively. Typically, a merger attachment will have hydraulic power supplied from the machine for a four-bar actuator and other related features such as a cross belt for crop discharge. The LVS for a merger attachment is generated by creating a 3D wireframe model of the displacements of the actuator and then adding another wireframe for the resulting motions of the cross belt; this is done by using a known CAD program. A schematic view of the combined primitives of the LVS is shown in FIG. 4C Mapping the 2D link movements of the actuator onto the side plane is the first step for generating the actuator portion of the LVS.

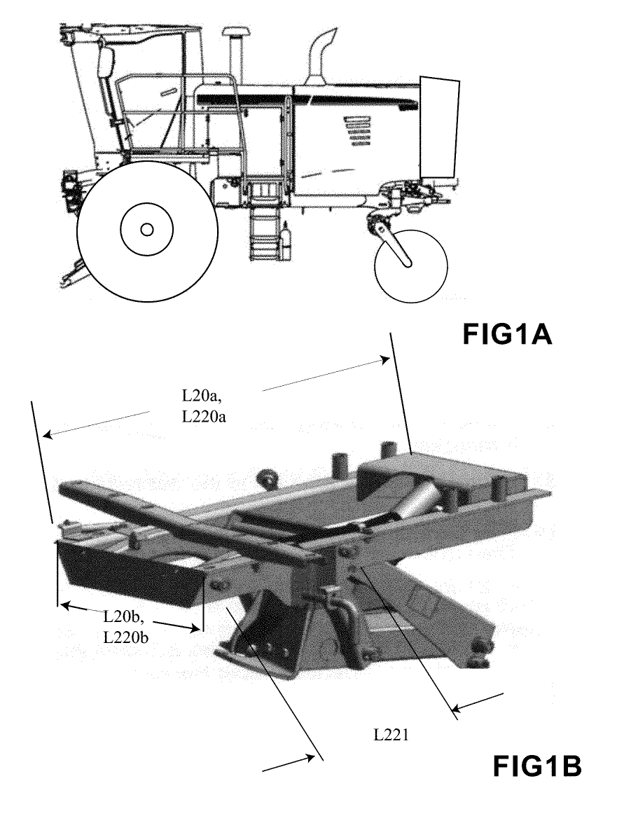

The actuation for cross-belt positioning movements of a merger is typically provided by a hydraulic-drive, four-bar linkage actuator component, which must be securely mounted to the machine chassis under the cab and between the front and rear wheels. An oblique view of the front of a typical actuator in the Up-state is shown in FIG. 1B. As can be seen, the top flange of the actuator mounting frame is a rectangle of width L20b and length L20a.

FIG. 4A is a schematic of the planes which create the primitive wireframe envelope for a typical four-bar actuator mechanism such as provided for a merger; this figure shows the left-side-oblique view of the wireframe looking generally in the forward-travel direction of the machine. This diagram depicts a portion of a wireframe-figure with 6 orthogonal faces and represents the space under the machine which must be available for mounting and operation of the actuator component.

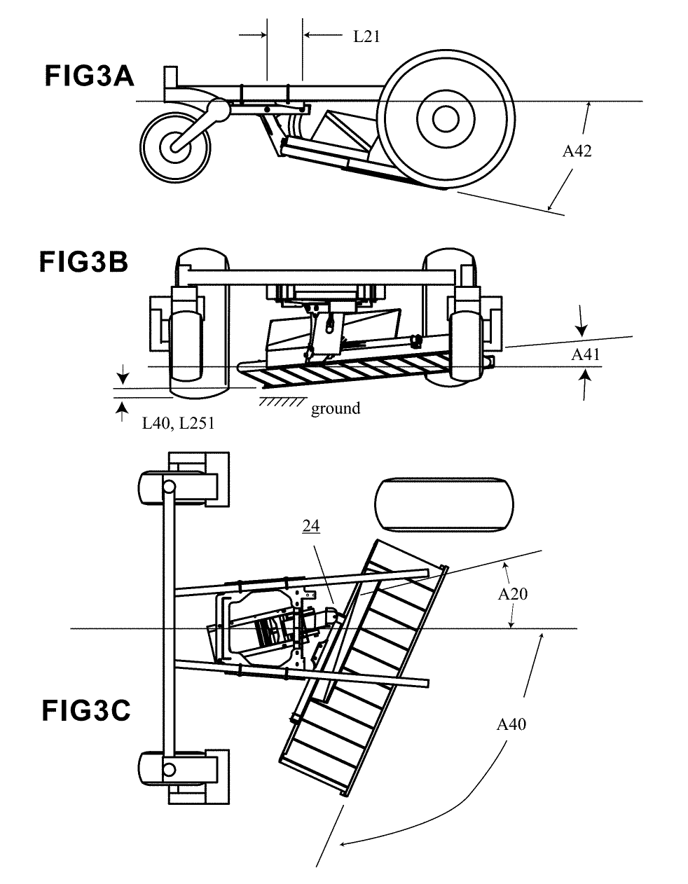

The actuator wireframe top plane, 50t, depicts the attachment plane for fixing the Up-Facing elements of the present invention. For most attachments as mounted, this plane lies horizontal, parallel to the ground and parallel to the mounting surfaces of the machine frame. As shown in FIG. 3C the edges of the actuator frame and plane 50s are oriented at an angle of A20, about 20 deg, to the machine axis. The top surface of this wireframe, plane 50t, which corresponds to the top of the actuator primitive wireframe, is shown in FIG. 4A with a length L22 and a width L23.

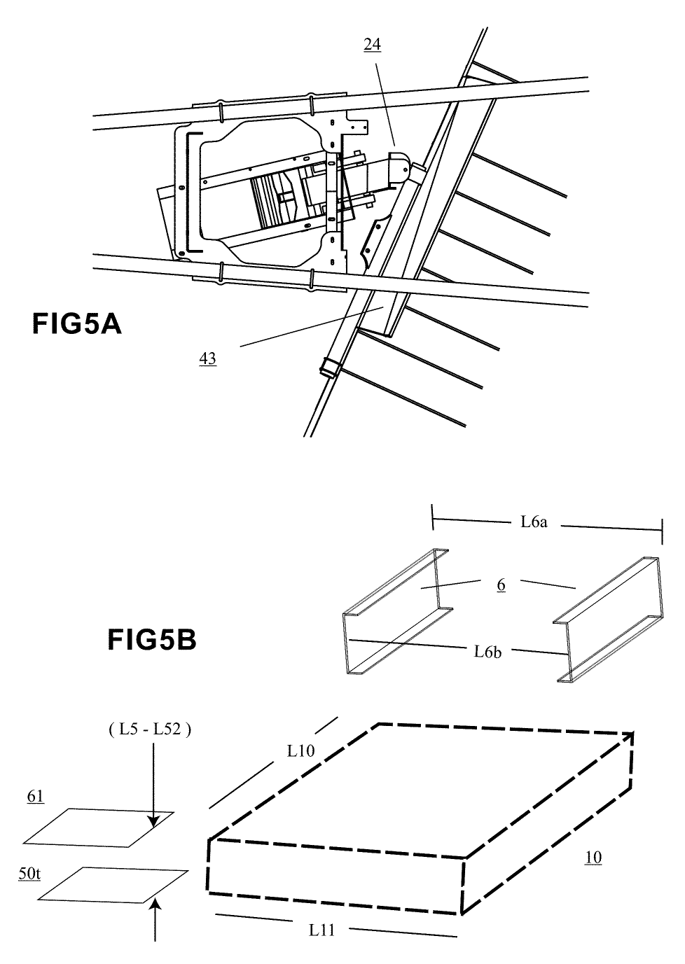

The actuator-wireframe front plane, 50f, represents the extreme limit of forward travel of the linkage-mechanism in moving into the Down state. This surface also reflects the approximate location of the swivel-joint-yoke-and-pin in the Down state. This yoke coupler, 23, which connects the cross-belt frame to the actuator, can also be seen in; FIG. 3C and FIG. 5A in this view the actuator is at full forward extension and plane 50f is modeled as vertical.

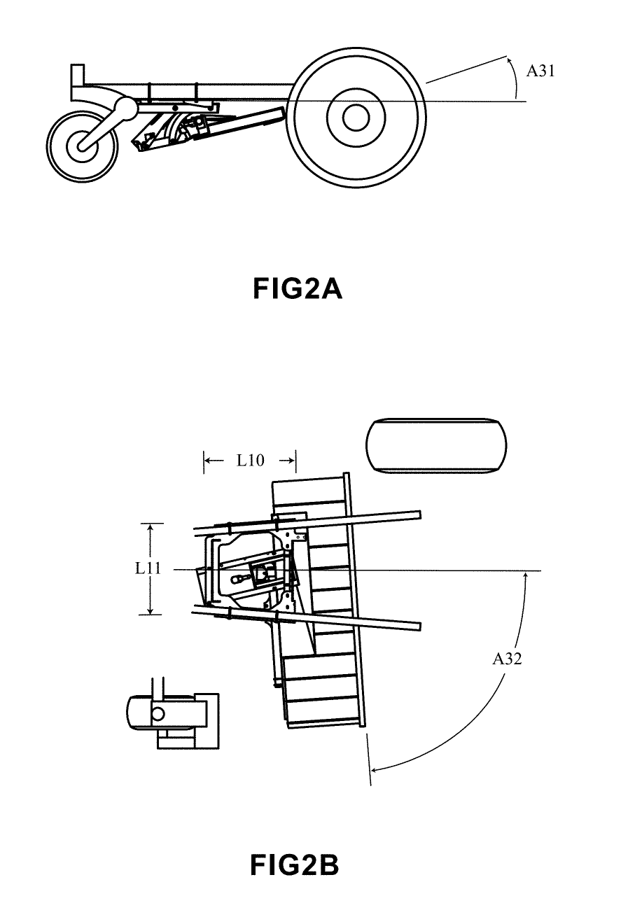

The vertical side plane, 50s shown in FIG. 4A represents one lateral face of the actuator primitive; the solid bars represent the linkage configuration in the Up state and the dashed bars represent them in the Down state. L21 represents the length of the fixed-linkage bar which is stationary and integrated into the actuator frame itself. The bars of a typical commercial actuator linkage can also be seen clearly in FIG. 1A, FIG. 2A and FIG. 3A.

The Up state of a merger attachment is the condition when the cross-belt component is moved to its uppermost position relative to the ground under the machine; this state is illustrated schematically in FIG. 2A. The Up state positions the cross-belt so that crop flow from the header (generally parallel to the machine axis) is not obstructed by the underside of the raised cross belt even at the maximum crop-flow capacity of the header. As shown in FIG. 2A, A31 is the longitudinal pitch angle of the bottom surface of the Up cross belt; typically, it is about 10 deg or upward. In Up-state harvesting operations, the crop-flow direction vector from the header is about −180 deg from the machine travel vector, i.e., generally parallel to the machine axis and the stream of as-cut plant material passes below the raised belt. Usually, the mounting mechanisms for the cross belt to the actuator provide links and swivels for some rotation of the belt orientation in the plane(s) parallel to the ground during the lowering process. This rotational movement in lowering is illustrated as from A32 (Up, FIG. 2B) to A40 (Down, FIG. 3C).

The cross-belt Down state is the condition when the cross-belt is deployed to its lower position for crop discharge just behind the right-front tire; this state is illustrated schematically in FIG. 3A. Typically the top surface of the Down cross belt at its proximal or receiving end falls slightly below the stream of crop flow from the header. Usually the orientation of the Down cross-belt axis, A40, is about −90 to −130 deg polar. The top surface of the Down belt may also have a small downward pitch angle in the vertical plane parallel to the machine axis, A42, i.e., belt surface is tilted forward and down about −5 to −15 deg polar. As can be seen in FIG. 3B, the Down belt-surface is rotated slightly upward in the vertical plane perpendicular to the machine axis, A41, about 1-5 deg. Some mergers may also have a short, articulated distal-tip portion of the cross belt, about 300 mm in length, which allows for automated compensation of the ground clearance of the outboard end of the Down belt in case of rough ground-terrain being detected by machine sensors and controls.

In the Down state, the cross-belt-top surface is positioned aft and slightly below the header discharge and its yaw- and roll-angles are optimized by the swivel yoke to capture and redirect crop flow entering in the axial direction (from the header) into a transverse direction for discharge above ground level just behind the right-front machine tire. The actuator yoke, 24, can be seen in FIG. 3C. In typical operation, with the machine moving forward (travel-direction vector=90 deg) at a velocity of about 2-5 m/sec, the cross belt redirects crop flow from the header toward the right side at an angle of about −90 deg to the machine-travel direction (A40).

The cross-belt component is also usually provided with an articulated, transversely-oriented shield-deflector to guide and retain axial crop flow onto the cross belt for discharge at its distal end. This shield is automatically raised_erected by links to the mounting fixtures of the cross belt in the Down state and similarly flattened against the top of the cross-belt in the Up state.

For a typical merger attachment, the cross belt is positioned between: (a) Up=crop flow passes below the belt and (b) Down=crop flow is captured, redirected and discharged in a direction generally perpendicular to the direction of machine travel behind the right front wheel of the machine.

The 3D wireframe envelope for the engaged, connected and coupled Down-Facing and Up-Facing elements is defined within the Unterface Primitive, (UP). The UP, (10) is illustrated in FIG. 5B as a multi-faceted, pseudo-rectangular wireframe which is extruded vertically downward between the bottom of the machine frame structure, or rails plane 61, and the top of the positioned actuator, plane 50t. Typically, the UP wireframe is defined by a set of vertical side faces; at least two are substantially parallel to the machine axis and two substantially perpendicular thereto. Profile sketches on the horizontal top- and bottom-faces of the UP wireframe serve to define to outline the Down- and Up-elements respectively of the present invention.

The length of the UP wireframe along the machine axis, L10, corresponds generally to about the length of the actuator frame; typically L10 is less than L20a since actuators are normally mounted off the machine centerline and oriented with their forward stroke in the vector direction 110-120 deg polar, i.e., at a small incident angle to the machine axis. The width of this wireframe, measured perpendicular to the machine axis as L11, is dictated by the frame-rail spacing and the size of the actuator top frame, L20b, which is to be attached to the Down element. L10 and L11 are illustrated in FIG. 2B.

The thickness aspect of the defining UP wireframe, indicates whether there may be a need for vertical spacing devices, for example high-strength flat washers, to achieve robust actuator mounting at exactly the proper ground spacing. Typically, plate material of thickness L70 in the range 3-10 mm is used to form all portions of the Down- and Up-elements. If the UP wireframe thickness, (L5-L52), exceeds about (2.1×L70), high-strength spacers with flattened_parallel end faces are required at one or more of the key mechanical interfaces.

The Down-Facing element is secured across_under the frame or sub-frame of the machine so that it positions the actuator, guides its vector movements and supports the in-service loads of crop flow upon the cross belt which it sustains. Known square-bend U-bolts of appropriate size and strength are used to secure this element to the channel or box sections which are common for machine-frame rails. The Down-Facing element is typically prepared by cutting from a single piece of flat-plate material; for structural steel such as A-36 plate, thickness in the range 6-10 mm is used and tear-out safety factors are applied to all zones of high stress (around corners and bolt holes).

In a CAD program, the Down-Facing element plate profile is defined by a “sketch” on the top face of the “wireframe”; this sketch corresponds to the polygonal area above the actuator which extends across the rails and along the machine axis beyond the actuator on both ends. For most applications the center area of this sketch is removed; usually the remaining band of material inside the area defined by L10×L11 is about 20 t wide, i.e., there is a peripherial ribbon of plate material about twenty plate thicknesses in width. For additional rigidity and strength of this element, stiffening ribs and/or other members are added (welded or bolted transversely) across either_both the rear_front portions of the plate profile. These stiffening members are fixed into the free space between the rails (above, on the up-side of the Down-Facing element plate). Front and rear transverse stiffeners are illustrated in the Example. These strengtheners prevent overstress and possible buckling_crippling failure of either transverse portion of the Down-Facing element.

In FIG. 4A and FIG. 4B, plane 61 shows a 2D profile polygon which corresponds to the profile of the Down-Facing element; this profile sketch has its longer aspect lengthwise and is centered along the machine axis. Typically, this polygon's width, L11, is about 105-115% wider, measured perpendicular to the machine axis, than the maximum outside dimension of the frame rails in the machine portion between the front and rear wheels. For parallel rails, the profile polygon width, L11, may be fixed along its length (between front and rear). For some machines, however, the rails or alternative sub-frame members, may not be parallel but rather oriented to one another at a small incident angle in the range 1-10 deg; for this case, profile width, L11, is graded lengthwise between a min. and max. value.

The length characteristic of the Down-Facing element profile is illustrated as L11, which is measured parallel to the machine axis. Generally, actuators are mounted offset and at a small angle oblique to the machine centerline; generally the actuator-motion-vector direction is about 110-120 deg. Spatial motions of the cross belt resulting from this actuator mounting also assure its proximal-end min. ground-clearance in the Down state and prevent obstruction of the header-discharge chute by the raised belt in the Up state.

Modern precision-ag machines are extremely complex and their unique attachments are custom-designed for each version; this is true even for different sizes of the same brand marque because of substantially different frame configurations under the cab. The main function of the Up-Facing element of the present invention is to provide a set of auxiliary, bolt-on mounting points for an attachment which was originally designed to bolt directly onto a particular foreign make model type of precision-ag machine. The combination of Up- and Down-Facing elements of the present invention forms a universal belly-mounting system which allows attachments to be used be usable with other machines.

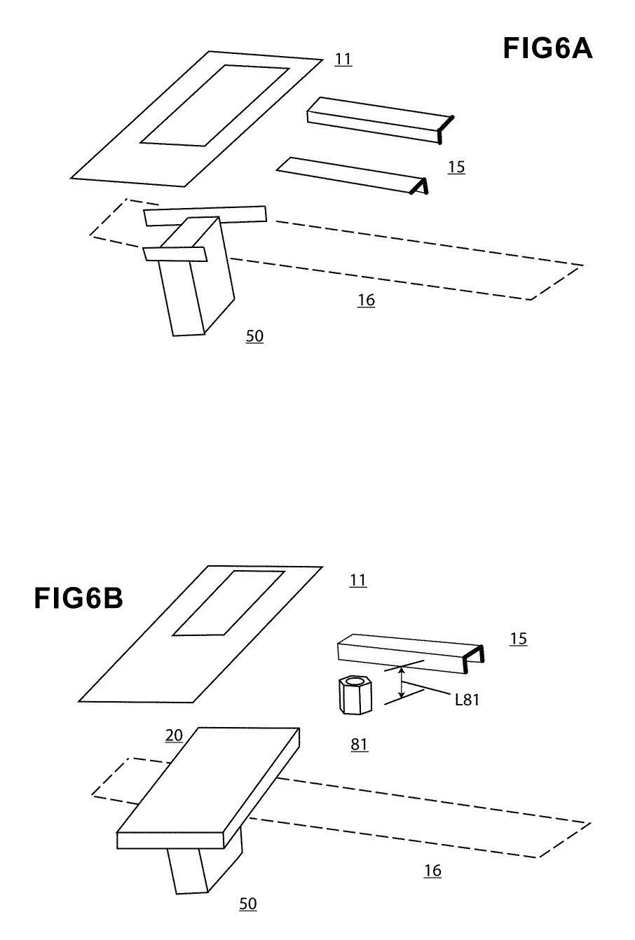

The profile for the Up-Facing element is defined by a sketch on plane 50t; this sketch includes one or more transversal components, 15, and spacers, 81, as shown in FIG. 6B. These components extend across the Down-Facing element and are bolted to it as well as to the attachment (top frame or flanges as shown in FIG. 6A).

In addition to mating the alternative mechanical-suspension points with the Down-Facing element, the Up-Facing element also frequently provides opportunities for more-accessible points to reposition industry-standard hydraulic-, electrical- and network-fittings and controls. Adding new-technology interfaces to replace legacy components can also contribute significantly to increasing operational reliability and reduction of maintenance costs by replacing the original proprietary, obsolete or inappropriate components and connections.

The Up-Facing element is initially bolted onto the attachment or merger which is, in turn to be joined to the machine with the Down-Facing element already in place. It also may be configured with an array of mechanical projections especially located, sized and shaped to interact with mating socket features incorporated into the Down-Facing element as they are being lifted and aligned together for bolting. These features, which come into play during the last few mm of the joining phase of the two elements, serve to urge the bolt holes into accurate registration without special factory lifting jigs and guides or a secondary line-reaming operation. Consider 2-3 strategically-located cone-like features extending upward from the Up-Facing element becoming seated into mating conical sockets opening downward. These features serve to guide the Up-Facing element into exactly the correct x-y position and angular orientation with the Down-Facing element under realistic conditions which might be experienced during an urgent mid-harvest, field changeover.

The optimal placement, physical form, final hole pattern and mechanical properties of the Up-Facing element(s) is dictated by each particular machine attachment combination and further defined by the thickness aspect of the resulting UP wireframe. Generally, if the actuator top plane provides a flat, rectangular flange with an array of holes for original mounting bolts, the Up-Facing element can be an additional beam-like component (15) transverse to the machine axis and long enough to bolt onto the ends of the peripheral ribbon during final mounting. A single, channel-form, transversal Up-Facing component is illustrated in the Example. In this case, the Up-Facing element is located at the front of the actuator where it extends between the frame rails; after bolting to the Down-Facing element it shares some of the loads related to the merger and crop handling.

FIG. 6A and FIG. 6B are a schematic illustration of two typical Up-Facing element configurations. The view point for both cases is toward the front of the machine and from above on the right side. The view shows the actuator as a wireframe box and the cross belt as a transverse, dashed outline.

FIG. 6A shows the case of a competitive commercial actuator suspended by short, transverse hanger flanges which extend upward above the top plane from its front and rear faces. This configuration is appropriate for an Up-Facing element composed of two transverse angle components, 15, fixed by bolts generally in the middle portion of the Down-Facing element.

FIG. 6B shows an actuator suspended by a rectangular top flange similar to the Example. In this configuration the transversal Up-Facing component is a channel, 15, which is bolted to the top of actuator frame on the front portion.

Engagement features guide the 3D positioning of the Down- and Up-Facing elements during field installation of an attachment or merger with simple lifting devices such as a jack or levers. Typical known engagement mating concepts for two flat_parallel surfaces include mating corner V-blocks and/or pin-and-funnel notch. When both elements become correctly positioned at contact, the critical bolt-holes are forced into alignment at the last stage of lifting; then the coupling components_fasteners are set and secured.

High-relief engagement features may also be integrated into the Down-Facing element and may project 2-10 mm. They also serve to distribute in-service loads on the attachment during typical use due to: machine vibrations, rough terrain, accidental light contact with ground-obstacles, and a plethora of crop-flow factors. Typical known engagement features include: corner-position reference block(s), cylinder-pin-and-notch alignment guide and tapered_pin-and-mating-socket positioning guide.

Typical engagement connections between the machine and the attachment include: mechanical links: cross-belt yaw angle control and minimum ground clearance, other; electrical power and/or signals: flashing-light “Cross Belt-ON” indicator, belt overspeed and overload warning lights, crop-flow blockage sensing, other;

hydraulic power and/or signals: belt drive, status indicator and auto ground-contact warning and cross-belt lift;
sensing network: monitoring of attachment characteristics and performance as a function of GPS coordinates in a field, i.e., correlation of crop flow and field coordinates, other.
   The Unterface system of this invention provides a method of interfacing between machine and attachment with standard connectors/data formats, whereby legacy attachments can be updated by adding advanced, off-shelf devices systems.

Coupling components are the known fasteners which accomplish mechanical connections between the attachment, the Down- and Up-Facing elements and the structure of the machine. These components are typically threaded fasteners, but other mechanical-coupling options may be used, especially for coupling the Up-Facing element to the attachment, i.e., rivets, welding, etc. All such joints are designed according to known engineering standards (SAE, ANSI, DIN, ISO) for the particular materials involved, specific fastener count_pattern and extreme service conditions for the attachment. Coupling components may include: std. high strength bolts, nuts, washers, studs, pins, other and special threaded fastening components with non-std. shape, size, thread parameters, other.

Because the present invention is intended to couple an attachment to machines which may lack automatic safety-shutdown interlocks between the machine and the attachment, this feature is included as an Unterface option. The first safety interlock senses: (a) the machine state, i.e., header Up/Down and power available for header and discharge belt and (b) the attachment state, i.e., Up_Down, hydraulic power available for cross-belt drive. This feature prevents starting the header and related crop discharge until the attachment is fully Up (no belt power) or fully Down.

The second safety interlock senses malfunctions and operational blockages of the cross-flow belt in the Down state, i.e., excessive forces building upon the transverse shield due to a crop-flow rate quantity mismatch between the machine discharge and the attachment cross-belt.

Unterface Kits. It is anticipated that variations of the present invention will be marketed in the form of particular matched sets of Up- and Down-Facing components, which have been: (a) sized and designed using the methods disclosed, and (b) precisely fabricated using appropriate materials and technics to allow connection and productive use of a particular attachment on a specific machine. Such kits will include user-friendly, illustrated technical guidelines (intended for an experienced mechanic or a dealer technician with modern training) detailing the various attachment and function adjustment processes (mechanical, electrical, hydraulic, network, other) along with required safety notices to be attached to the assembled Unterface adapter, to the attachment and to the machine, especially its controls. It is envisioned that some kits will be heavy enough to justify packaging and shipment in the form of a wrapped pallet or completely encased within a strong crate. Either embodiment would require delivery and handling by a fork-lift; as appropriate, such kits will also be packaged along with with certain unique jigs_fixtures to facilitate safe, accurate and rapid mounting and attachment/calibration in the field or in a shop.

“MacJohn” Unterface Example. This example illustrates application of the Unterface method to a specific commercial machine (JD W235 windrower) for mounting of a foreign, third-party attachment (MacDon DWA, which was originally designed for various MacDon headers, augers, drapers and rotary disc). The result of using the method is the “MacJohn” apparatus for coupling the particular machine and specific attachment.

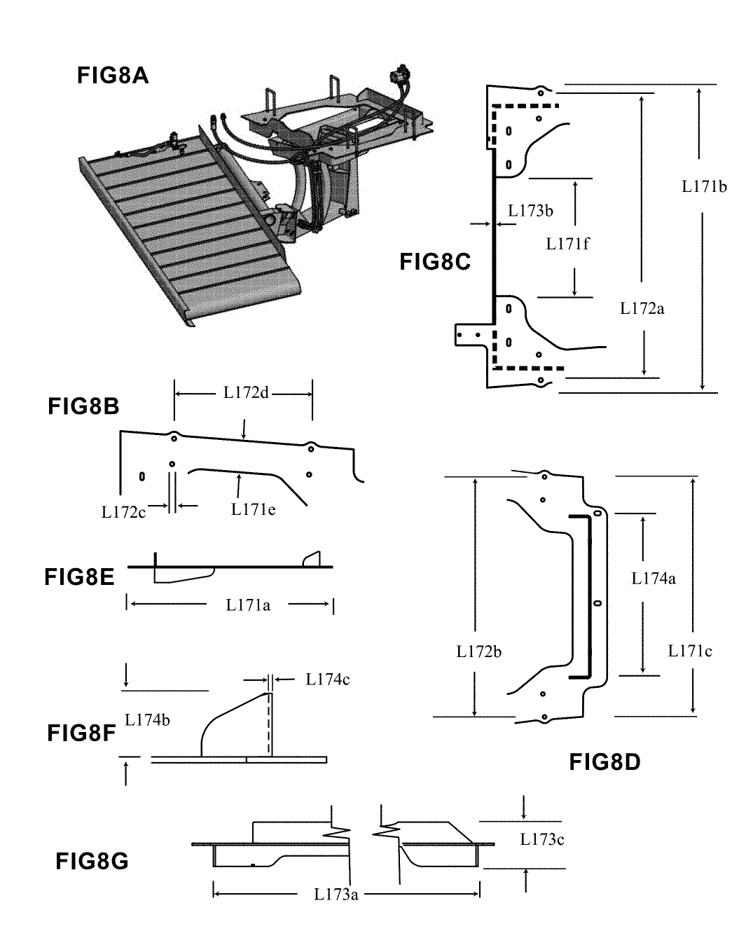

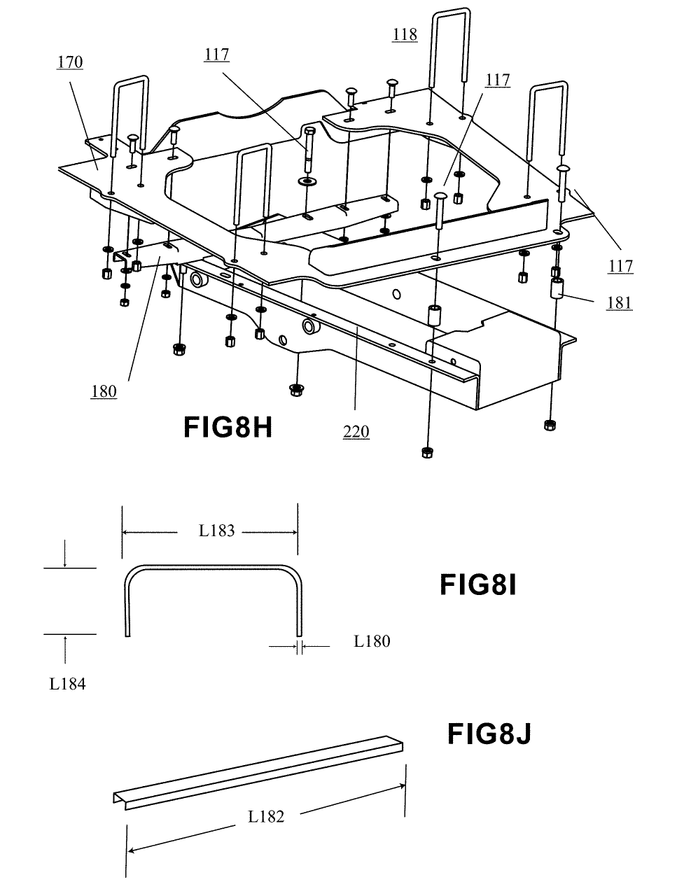

FIGS. 8A-8C show schematic drawings of the “MacJohn” Unterface embodiment for this combination. TABLE 2 provides some selected technical data for the selected machine and particular attachment.

The initial step in this exemplary Unterface adaptation process is to map the operational characteristics of the DWA into a 3D CAD model to locate the exact x-y-z coordinates of the top edges of the actuator (and all of its mounting-bolt holes) as well as its orientation re the machine axis as correctly mounted .AND. in the Down state. This merger loft model will define: L220, L221, L222, L223, L251, L252, L253 and L260. The height difference, between the top plane of the actuator, 50t, and the bottom plane of the machine frame rails, 61, is the key factor which fixes the thickness aspect of the Unterface primitive, 10.

Knowing the general area of the mounting zone, the pseudo-rectangular wireframe of the Unterface Primitive (UP) can be extruded upward from the actuator frame .OR. downward from the frame rails to fill the available space allowable for the engaged elements. The resulting UP envelope between planes 50t and 61 defines: (a) the 3D space within which the Down- and Up-Facing elements must fit and (b) how far the edges of plane 50f must lie to the rear of the axis of the machine front wheels.

Assuming that the thickness aspect of the resulting UP is at least 20 mm, it will be possible to use the Unterface system for mounting the DWA with “MacJohn” elements fabricated from flat plate in the thickness range 6-10 mm. The front-transversal component of Up-Facing element is a steel channel which is needed to support the front of the actuator; the rear portion of the Up-Facing element includes an array of unique stabilizing spacers 81, which match the height of the transversal component.

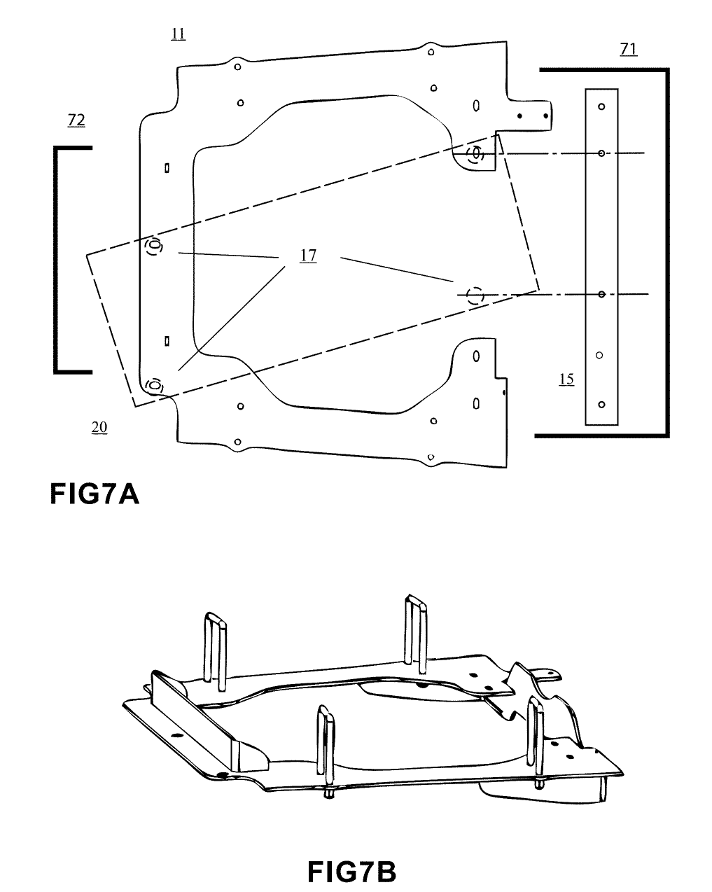

Details of the “MacJohn” Down- and Up-Facing elements are shown in FIG. 7A and FIG. 7B; an external view of the MacDon DWA is shown as FIG. 1B. The DWA actuator frame is about 1.8 m long and the full stroke of its four-bar linkage amounts to about 600 mm; thus, the actuator primitive length for this attachment, L222, is typically more than 2.3 m. Also, as can be seen in FIG. 1B, the hydraulic-cylinder base protrudes about 20-50 mm above its top-rim flange and special, thick-walled, high-strength, end-ground tubular spacers are needed for the bolts which fasten it to the Down-Facing element.

The “MacJohn” Down-Facing element profile is defined by a 4-sided UP primitive having its ends perpendicular to the machine axis; the two end portions are parallel and about 1.3 m and 1.5 m long, respectively. The non-parallel lateral portions are each about 1.3 m long and the incident angle between them is about 9 deg; they are centered lengthwise about the machine axis. The thickness aspect of this primitive is about 20-30 mm; spacers are used on the rear bolts which connect between 11 and 20; no spacers are needed on 15, the Up-facing transversal component(s) (in this case, is a single, inverted-U channel of equal height extending across the front of the actuator and anchoring its two front bolts to 11. The final profile of the main portion of Down-Facing element, complete with the rationalized bolt-hole pattern is shown in FIG. 7A.

The main portion of the Down-Facing element, 11, is defined by U-shaped profile sketch on plane 61; this component is cut from flat A36 steel plate about 9 mm thick and the center is removed to leave a peripheral ribbon of width about 180-190 mm. Because of the particular loadings which occur in heavy use, transverse stiffener-members, 71 and 72 are welded across both ends of this element. For clarity, both these elements are shown in FIG. 7A in vertical profile and the profiles are laterally displaced from their actual location in the final weldment. As shown in FIG. 7B, component 72 is fillet welded on the top side and 71 is fillet welded to the bottom side. Component 71 extends across the front-end portion of 11 to limit its deflections under torsion; this placement leaves a narrow transverse space between the actuator frame, 20 and 71 for the Up-Facing element, 15.

The profile of the Up-Facing element of the MacJohn is defined by a sketch on plane 50t; the main portion is a transversal component, 15, which connects between 11 and 20. As shown in FIG. 6B, this part is an inverted U- or C-profile structural channel (placed U-opening downward) which is formed from stock of sufficient strength, thickness, width and height to carry maximum merger loads with at least a TYS safety factor of 2. Typically, this member is formed from A36 stock, has web thickness in the range 3-8 mm; the transversal-component width is about 70-150 mm and the height (vertical dimension of section) matches the spacers required at the rear of the actuator. This component extends across the front portion of 11 and between the arms of 71.

The MacDon Double Windrow Attachment is designed to mount on their M-series machines such as the M205 Self-Propelled Windrower. This unit is built on a unique box frame which supports the cab, engine and under carriage. For compatibility with multiple machines, the DWA mounting parts include a special-shape support weldment with multiple holes, slots and an integrated, threaded post fastener to secure it to the actuator. For the several prescribed target MacDon models versions, this component attaches to the crossbar surface of each distinctive box frame below the engine mounts. While this part (176060) was tested with prototypes of the Down-Facing element of the Unterface, the openings and length-variations of section stiffness are recognized as a source of stress concentration which—as loaded in the transversal Up-Facing component—may lead to undamped resonant vibrations and premature development of fatigue cracks. Unterface and MacJohn design practice for conservative transversal section area has led to specification of several A36 alternate Up-Facing transverse components free of geometric discontinuities and other stress raisers.

The assembled pair of Unterface elements is designed to support: (a) axial forces on the shield and cross-belt due to max crop flow input from header, (b) torsional loads (transverse plane) on cross-belt swivel due to crop-flow re-direction, (c) bending loads (vertical plane) on cross-belt swivel due to rough field terrain, and (d) bending loads (horizontal plane) on cross-belt swivel due to accidental, brief ground contact. These goals are achieved by careful adjustment of the elastic rigidity characteristics (flexure and torsion) of the two transverse stiffeners, 71 and 72, i.e., their elastic and shear moduli, and section profile/area. The geometric rigidity factors of these components include specific-axis moments of inertia, and specific torsional-rigidity multipliers. Further, continuous fillet MIG welds are used on both sides where they are joined perpendicular to the surface of the main-plate member.

Sets of custom-shaped, high-strength U-bolts provided for MacJohn Unterfaces are selected from types 1,2 and 5 (the particular shape depending on the form of machine frame members) and coined/flattened across the contact surface; the material may be of either grades 5 and 8 depending on the attachment and intended field operations.

TABLE 1
Unterface Features & Indicia

| | | |
|---|---|---|
| Indicia | Feature or Characteristic, units | Range |

1-9 typical machine

| | | |
|---|---|---|
| L1 | wheelbase, m | 3-5 |
| L2 | track, m | 2-3.5 |
| L5 | frame_rail bottom plane ht, m | 0.9-1.5 |
| L6a | min. frame_rail outside width, m | ~0.8 |
| L6b | max. frame_rail outside width, m | ~2 |

10-19 Unterface elements

| | | |
|---|---|---|
| 10 | Unterface primitive for Up&Down engaged | |
| | elements | |
| 11 | Unterface Down-facing element | |
| L10 | Length of Down-facing element, parallel | 1.1-2.5 |
| | machine axis, m | |
| L11 | Width of Down-facing element, perp. | 1.1-2.5 |
| | machine axis, m | |
| 15 | Unterface Up-facing element | |
| 16 | cross belt | |
| 17 | high-strength inter-element fasteners | |

20-29 Actuator

| | | |
|---|---|---|
| 20 | actuator mounting frame | |
| L20a | actuator mounting frame length, m | 1.1-2.5 |
| L20b | actuator mounting frame width, m | 1.1-2.5 |
| L21 | length of actuator fixed-link, m | 0.2-1 |
| L22 | actuator primitive length, m | 0.5-2 |
| L23 | actuator primitive width, m | 0.2-2 |
| A20 | actuator mounting frame yaw angle, deg | ~0-30 |
| 24 | actuator yoke | |

30-39 Cr_Belt-Up

| | | |
|---|---|---|
| 30 | Up cross belt | |
| A31 | Up cross belt surface pitch angle, deg | ~5-30 |
| A32 | Up cross belt axis yaw angle, deg | ~75-120 |
| L33 | cross belt length, mm | |
| L34 | cross belt width, mm | |

40-49 Cr_Belt Down

| | | |
|---|---|---|
| 40 | Down cross belt | |
| A40 | Down cross belt axis yaw angle, deg | ~75-120 |
| A41 | Down cross belt surface roll angle, deg | ~0-20 |
| A42 | Down cross belt surface pitch angle, deg | ~0-30 |
| 43 | cross belt shield | |

50-59 Merger LoftVol Prim

| | | |
|---|---|---|
| 50 | merger actuator primitive wireframe in LVS | |
| 50f | front plane of actuator primitive | |
| 50t | top plane of actuator primitive | |
| 50s | side plane of actuator primitive | |
| 51 | cross-belt 3D primitive in LVS | |
| L51 | cross-belt Down-state ground clearance, mm | 50-200 |
| L52 | ht. of actuator wireframe top face 50t, above | 0.9-1.2 |
| | ground, m | |
| L53 | min. ground clearance of actuator wireframe | 0.2-0.8 |
| | bottom, m | |

60-69 Machine Frame

| | | |
|---|---|---|
| L60 | machine frame rail ground clearance, m | 0.9-1.2 |
| 61 | plane of bottom of machine frame rails | |

70-79 Down_facing

| | | |
|---|---|---|
| 70 | Down-facing element | |
| L70 | plate thickness, mm | ~3-10 |
| 71 | Down-facing front stiffener | |
| 72 | Down-facing rear stiffener | |

80-89 Up facing

| | | |
|---|---|---|
| L80 | transversal component thickness, mm | ~3-10 |
| 81 | stabilizing spacer component | |
| L81 | spacer length, mm | ~10-200 |

JD W200

| | | |
|---|---|---|
| L105 | frame_rail bottom plane height, m | ~1.13 |
| L106a | min. frame_rail outside width, m | ~1.26 |
| L106b | max. frame_rail outside width, m | ~1.36 |

MacDon DWA

| | | |
|---|---|---|
| 220 | actuator mounting frame | |
| L220a | actuator mounting frame length, m | ~1.35 |
| L220b | actuator mounting frame width, mm | ~530 |
| L221 | length of actuator fixed-link, mm | ~500 |
| L222 | actuator primitive length, m | 1.07-1.22 |
| L223 | actuator primitive width, mm | 360-480 |
| L251 | cross-belt Down-state ground clearance, mm | 50-80 |
| L252 | ht. of actuator wireframe top face, 50t, above | ~1.04 |
| | ground, m | |
| L253 | min. ground clearance of actuator wireframe | ~230 |
| | bottom, mm | |

TABLE 2
Data for Exemplary Machine, Attachment and Application

| | | |
|---|---|---|
| Feature | Characteristic Parameter | Range |

MacJohn Unterface Data

| | | |
|---|---|---|
| 117 | high-strength inter-element fasteners | M6-M14 |
| 118 | set of machine-matched U-bolts | M6-M14 |
| 170 | Down-facing element | |
| L171a | main-plate length, mm | ~1401 |
| L171b | main plate forward width, mm | ~1450 |
| L171c | main plate aft width, mm | ~1276 |
| L171d | main plate thickness, mm | ~9 |
| L171e | main plate typical peripheral ribbon width, | ~184 |
| | mm | |
| L171f | main plate forward opening width, mm | ~572 |
| L172a | forward-outside U-bolt hole span, mm | ~1379 |
| L172b | aft-outside U-bolt hole span, mm | ~1278 |
| L172c | U-bolt hole diam, mm | ~10 |
| L172d | spacing between forward and aft U-bolts, | ~658 |
| | axial | |
| L173a | forward stiffener width, mm | ~1270 |
| L173b | forward stiffener thickness, mm | ~9 |
| L173c | forward stiffener height, mm | ~213 |
| L174a | aft-stiffener width, mm | ~872 |
| L174b | aft-stiffener height, mm | ~100 |
| L174c | aft-stiffener thickness, mm | ~9 |
| 180 | Up-facing element, channel component, | |
| | 75 × 40 to 100 × 50 | |
| 181 | spacer components for Up-facing element | |
| L181 | Up-facing spacer component length, | ~25 |
| | sq-ground ends, mm | |
| L180 | Up-facing channel component thickness, | ~6-9 |
| | mm | |
| L182 | Up-facing channel component length, mm | ~1102 |
| L183 | Up-facing channel component width, mm | ~76 |
| L184 | Up-facing channel component depth, mm | ~25 |

1-3. (canceled)

4. An Unterface system for mounting and safe operation . . .

5. The system according to claim 4. adapted to mount said attachment

6. A system according to claim 5. adapted to mount said attachment with a customized

7. A system according to claim 5. wherein the sub-assembly of the attachment

8. A system according to claim 5. wherein the sub-assembly of the Down-Facing element, the attachment and the Up-Facing element

9. An enhanced system of claim 5 wherein the improvement additions to said Down-Facing element comprise:

10. An enhanced system of claim 9 wherein the further improvement additions to said Down-Facing element comprise:

11. An enhanced system of claim 5 wherein the improvement additions to said Up-Facing element comprise one or more of the following

12. An enhanced system of claim 10. wherein the improvement additions to said Up-Facing element comprise one or more of the following:

13. An Unterface system for mounting and safe operation of a MacDon DWA

14. A kit of matched Unterface components

4. An Unterface system for mounting and safe operation of a particular functional attachment, with machine pairing for full attachment operation, behind the front wheels and mounted under a designated precision-agriculture machine having 2 or more wheels and substantially laterally centered respecting its longitudinal centerline, comprising:
(a) a first Down_facing element removably attachable to said machine and having one or more separable components;
(b) said first element provided with an specific array of known mateable engagement features, adapted to attach and position said attachment into a particular location and orientation on said machine;
said first element being further provided with one or more known industry-standard transfer connections for supplying selected motive power(s) and providing an operator interface for input of selected control signals to drive, control and monitor the allowable 3D orientations/positions and functions of said attachment from from operator's seat of said designated machine;
(d) a second Up_facing element configured to interconnect removably between said first element and said attachment;
(e) said second element being provided with an array of mateable engagement features designed to mate with said specific engagement features of said first element, whereby precise positioning and alignment to said machine is achieved at their mechanical joining;
(f) said second element being further provided with an array of known industry-standard transfer connections designed to mate with corresponding connections of said first element and removably coupled to power and control interconnection fittings of said attachment;
(g) said connections adapted to interlink with transfer connections of said first element whereby, upon mechanical joining said first and second elements and union of the several provided transfer connections, the attachment is supported, powered and controlled from said machine;
(h) the preferable size, shape, and mechanical properties of each of said elements is defined according to accepted engineering design and industry safety practices considering modeling results for probable stresses generated by typical operation of the attachment;
(i) the mechanical properties, location, count and configuration of said array of engagement features being defined to permit a set of predefined 3D movements of the attachment and its temporary elastic-flexural bending under worst-case operational loads;
(j) the type, size and 3D location_orientation of said array of transfer connections being defined primarily by said attachment and its maximum in-service loads, power requirements and x-y-z travel limits as mounted on the machine;
(k) whereby, upon coupling of said engagement features and connections along with said first and second elements, the resulting combination with said designated machine provides integrated mechanical, hydraulic, electrical and wireless network connections for power transfer and function-control signals between said particular machine and said particular attachment beneath said machine and between its front and rear wheels.

5. The system according to claim 4. adapted to mount said attachment between the front and rear wheels of a 4-wheeled machine at a location which assures its full range of functions and without mechanical interference with any machine part.

6. A system according to claim 5. adapted to mount said attachment with a customized orientation defined by the yaw axis angle of the actuator mounting being different from (A20).

7. A system according to claim 5. wherein the sub-assembly of the attachment and the Up-Facing element provides a lateral displacement of the attachment a distance of 50 to 500 mm from the longitudinal centerline of said machine.

8. A system according to claim 5. wherein the sub-assembly of the Down-Facing element, the attachment and the Up-Facing element provides a lateral displacement of the attachment a distance of 50 to 500 mm from the longitudinal centerline of said machine.

9. An enhanced system of claim 5 wherein the improvement additions to said Down-Facing element comprise:
(a) adding a known wireless high-speed internet connection along with a known dynamic GPS locator;
(b) adding a known programmable flat, touch-screen display system adjacent operator's seat networked together with said internet connection and said GPS locator;
whereby the operator can visualize the instant location and movements of the attachment within a selected crop plot.

10. An enhanced system of claim 9 wherein the further improvement additions to said Down-Facing element comprise:
known systems networked together with said touch-screen display to provide real-time access by the machine operator to currently-updated databases for designated characteristics of selected crop(s) and particular soil characteristics of the selected plot(s) including one or more of:
(a) display of photographic-images of selected crops, said images being either satellite or drone sourced, as recorded and stored earlier in the specific crop season;
(b) display of data graphics depicting time trends of soil moisture across the entire plot area as recorded earlier in the specific crop season; and
(c) display of data graphics depicting cumulative applications of fertilizers, pesticides or herbicides across the entire plot area as recorded earlier in the specific crop season;
whereby the machine operator can optimize machine travel speed, attachment positioning and its operational parameters to suit instant conditions within selected zones of the selected crop plot.

11. An enhanced system of claim 5 wherein the improvement additions to said Up-Facing element comprise one or more of the following:
(a) a known system to provide real-time measurement of moisture in standing or cut crop while the machine is traveling forward at speeds of 0.05 to 0.5 m/sec with the attachment raised;
(b) a known system to provide real-time measurement of moisture in cut crop being delivered to the lowered attachment cross belt while the machine is traveling forward at speeds of 0.05 to 0.5 m/sec; and
(c) a known system to provide dynamic, real-time measurements of the mass flow rate of crop flow across the cross belt surface while the machine is traveling forward at speeds of 0.05 to 0.5 m/sec;
whereby the machine operator can optimize machine travel speed, attachment positioning and its operational parameters to suit instant conditions within selected zones of the selected crop plot and optionally command capture of the data streams for later analysis.

12. An enhanced system of claim 10. wherein the still further improvement additions to said Up-Facing element comprise one or more of the following:
(a) a known system to provide real-time measurement of moisture in standing or cut crop while the machine is traveling forward at speeds of 0.05 to 0.5 m/sec with the attachment raised;
(b) a known system to provide real-time measurement of moisture in cut crop being delivered to the lowered attachment cross belt while the machine is traveling forward at speeds of 0.05 to 0.5 m/sec; and
(c) a known system to provide dynamic, real-time measurements of the mass flow rate of crop flow across the cross belt surface while the machine is traveling forward at speeds of 0.05 to 0.5 m/sec;
whereby the machine operator can optimize machine travel speed, attachment positioning and its operational parameters to suit instant conditions within selected zones of the selected crop plot and optionally command capture of the data streams for later analysis.

13. An Unterface system for mounting and safe operation of a MacDon DWA attachment, with machine pairing for full attachment operation, between front and rear wheels and substantially under a John Deere W200 machine or similar JD model, comprising:
(a) a first Down facing element removably attachable to said machine and having one or more separable components;
(b) said first element provided with an array of known mateable engagement features, adapted to attach and position said attachment into a particular location and orientation on said machine;
(c) said first element being further provided with one or more known industry-standard transfer connections for supplying selected motive power(s) and providing an operator interface for input of selected control signals to drive, control and monitor the allowable 3D orientations_positions and functions of said attachment from operator's seat of said designated machine;
(d) said first element formed of A-36 steel plate and having the general dimensions defined by L10, L11 and L80, said first element including one or more components as further defined by dimensions provided in FIG. 8A to FIG. 8J and TABLE 2;
(e) a second Up facing element configured to interconnect removably between said first element and said DWA attachment;
(f) said second element being provided with an array of mateable engagement features designed to mate with corresponding engagement features of said first element, whereby precise positioning and alignment to said machine is achieved at their mechanical joining;
(g) said second element being further provided with an array of known industry-standard transfer connections designed to mate with corresponding connections of said first element and removably coupled to power and control interconnection fittings of said DWA attachment;
(h) said connections adapted to interlink with transfer connections of said first element whereby, upon mechanical joining said first and second elements and union of the several provided transfer connections, the DWA attachment is supported, powered and controlled from said machine;
(i) said second element including one or more components as further defined by sketches on plane 50t along with dimensions provided in FIG. 8A to FIG. 8J and TABLE 2
(j) the preferable size, shape, and mechanical properties of each of said elements is defined according to accepted engineering design and industry safety practices considering modeling results for probable stresses generated by typical operation of the attachment;
(k) the mechanical properties, location, count and configuration of said array of engagement features being defined to permit a set of predefined 3D movements of the attachment and its temporary elastic-flexural bending under worst-case operational loads;
(l) the type, size and 3D location orientation of said array of transfer connections being defined primarily by said attachment and its maximum in-service loads, power requirements and x-y-z travel limits as mounted on the machine;
(m) whereby, upon coupling of said engagement features and connections along with said first and second elements, the resulting combination with said JD machine provides integrated mechanical, hydraulic, electrical and network connections for power transfer and function-control signals between said particular machine and said DWA attachment mounted generally beneath said machine between its front and rear wheels.

14. A kit of matched Unterface components adapted to allow mounting of a selected attachment intended for crop cultivation, management or harvesting to a particular designated ag machine comprising:
(a) first Down_Facing element having one or more separable components, attachable to said machine and adapted, by an array of engagement features, to connect, drive and control the allowable 3D orientations_positions and functions of said attachment from said designated machine, said first element being still further adapted to provide one or more industry-standard transfer connections for supplying selected motive power and providing control signals to said attachment in conjunction with said designated machine;
(b) second Up_Facing element configured to mate with said first element and including an array of matched transfer connections securely affixed to upward-facing structures of said particular attachment and adapted to mate with said connections and with connect with said engagement features of said first element whereby, upon engagement, the attachment is supported, powered, controlled and monitored from said machine;
(c) a set of selected of mechanical fasteners and engagement features adapted for temporary coupling of said elements together for safe use of said attachment in field work;
(d) sets of selected fasteners and fixtures adapted to attach said said first element to said machine and said second element to said attachment, respectively;
(e) a set of selected circuits, fasteners, adapters, connectors, valves, terminals, instruments and sensing components especially adapted for the said machine-attachment combination whereby unique, non-standard fittings/hardware/formats provided native on each, respectively, is recast into industry-standard fittings/hardware/formats to facilitate power transfer, network communication and safety/control functions between said machine and said attachment;
(f) a set of print documents containing illustrated technical guidelines and links to web info and videos covering: (f1) assembly of the Unterface elements to said machine and to said attachment, (f2) inter-connection of the recast engagement features and (f3) verification of the operational power, safety and cross-belt positioning controls as well as the various network-monitoring features; and
(g) a set of weatherproof, adhesive-backed labels covering safe operation of the attachment and warnings related to typical hazards in agricultural operations, said labels to be attached in prominent locations on/in the machine cab to be easily visible to every operator.
